(19) United States
(12) Patent Application Publication
(10) Pub. No.: US 2016/0349249 A1
(43) Pub. Date: Dec. 1, 2016

(54) HIGH-SENSITIVITY LATERAL-FLOW IMMUNOCHROMATOGRAPHIC CHIP USING ENZYME-MIMIC INORGANIC NANOPARTICLES AND DETECTION METHOD USING SAME

(71) Applicant: POSTECH ACADEMY-INDUSTRY FOUNDATION, Pohang-si, Gyeongsangbuk-Do (KR)

(72) Inventors: JUNSANG DOH, POHANG-SI, GYEONGSANGBUK-DO (KR); JINWOO LEE, SEOUL (KR); MIJU KIM, BUSAN (KR); MINSU KIM, GURI-SI (KR)

(73) Assignee: POSTECH ACADEMY-INDUSTRY FOUNDATION, Pohang-Si, Gyeongsangbuk-Do (KR)

(21) Appl. No.: 15/113,807

(22) PCT Filed: Jan. 15, 2015
(86) PCT No.: PCT/KR2015/000431

§ 371 Date: Jul. 22, 2016

(30) Foreign Application Priority Data
Feb. 4, 2014 (KR) 10-2014-0012782

(51) Int. Cl. G01N 33/543; G01N 33/553

(52) U.S. Cl. CPC G01N 33/54346; G01N 33/553

(57) ABSTRACT

This invention relates to a method of manufacturing a lateral-flow immunochromatographic chip using the protein enzyme-simulating catalytic activity of inorganic nanoparticles and, more particularly, to a method of fixing an antibody, which is capable of detecting an analyte, to nanoparticles of iron oxide (Fe₃O₄) and platinum (Pt), and amplifying a chromogenic signal using an enzyme-substrate reaction of the resulting nanoparticles, and a lateral-flow immunochromatographic chip manufactured using the same. The lateral-flow immunochromatographic chip is used to manufacture a bio-chip for detecting component materials with high sensitivity. The lateral-flow immunochromatographic chip according to this invention includes a conjugation unit that includes enzyme-mimic inorganic nanoparticles labeled with a detection antibody.

### TECHNICAL FIELD

The present invention relates, in general, to a method of manufacturing a lateral-flow immunochromatographic chip using protein enzyme-simulating catalytic activity of inorganic nanoparticles and, more particularly, to a method of fixing an antibody, which is capable of detecting an analyte, to inorganic nanoparticles, such as iron oxide (Fe₃O₄), platinum (Pt), graphene oxide, and V₂O₅, and amplifying a chromogenic signal using an enzyme-substrate reaction of the resulting nanoparticles, and to a lateral-flow immunochromatographic chip manufactured using the same. The lateral-flow immunochromatographic chip may be used to manufacture a bio-chip for detecting component materials with high sensitivity.

### BACKGROUND ART

A bioassay using a flow immunochromatographic chip (lateral flow immunoassay chip) is a technology for simply and rapidly detecting an analyte in body fluid. Bioassays have occupied a major part of the diagnostic test market for a long period of time. A labeling process using gold nanoparticles as a chromogenic material has been most frequently used to simply perform diagnosis with the naked eye in practical applications using the characteristic of the gold nanoparticles, which exhibit a red color due to the intrinsic plasmon phenomenon of the gold nanoparticles. However, the biggest problem with the detection system using the aforementioned process is the dependency on an analyte that is present in a large amount in body fluid due to the low analysis sensitivity. This limited ability to detect small amounts of materials prevents the range of application of the current detection system, which is an initial countermeasure means for responding to disease, from expanding.

Therefore, Korean Laid-Open Patent Application No. 2013-0090174 discloses a bio-sensor that includes a reaction unit and a measurement unit. The reaction unit includes a first fusion material, which includes a reaction material, which is capable of being combined with a target material, and a light-absorbing material, which is combined with the reaction material. The measurement unit includes a second fusion material including a detection material, which is capable of being combined with the target material or the reaction material that is combined with the target material, and a fluorescent material combined with the detection material. The second fusion material is fixed to a support.

Further, Korean Laid-Open Patent Application No. 2010-118550 discloses a method of amplifying a signal in a lateral-flow assay. The method includes combining a conjugation substance with an analyte, the conjugation substance including a first antibody or a first specific combination material, which is capable of being specifically combined with a first epitope or a first combination portion (ligand) of the analyte, and gold nanoparticles combined with the first antibody or the first specific combination material; combining the analyte, which is combined with the conjugation substance, and a fixed second antibody or second specific combination material, which is specifically combined with a second epitope or a second combination portion (ligand) of the analyte; and adding gold ions and a reducing agent to induce a reaction.

However, this method has a drawback in that a separate fluorescent material or a measurement device that is specialized for the purpose of detecting a signal is required in order to improve the low assay characteristic of the gold nanoparticles.

### DISCLOSURE

### Technical Problem

Accordingly, the present invention has been made keeping in mind the above problems occurring in the related art, and an object of the present invention is to provide a novel high-sensitivity lateral-flow immunochromatographic chip using inorganic nanoparticles.

Another object of the present invention is to provide a novel high-sensitivity lateral-flow immunochromatographic chip for amplifying a signal.

A further object of the present invention is to provide novel particles which are combined with a target material in a conjugation unit of a lateral-flow immunochromatographic chip and then move to a reaction unit to be fixed to thus exhibit a detection signal, and which are reacted with a chromogenic substrate in the reaction unit to amplify the signal.

### Technical Solution

In order to accomplish the above objects, the present invention provides a lateral-flow immunochromatographic chip that includes a conjugation unit. The conjugation unit includes enzyme-mimic inorganic nanoparticles that include a detection antibody.

The lateral-flow immunochromatographic chip according to the present invention includes the conjugation unit that includes the enzyme-mimic inorganic nanoparticles labeled with the detection antibody, and the protein enzyme inorganic nanoparticles are combined with a target material to be fixed to a reaction unit and are then reacted with a chromogenic substrate to amplify a detection signal.

In order to accomplish the above objects, the present invention also provides enzyme-mimic inorganic nanoparticles which are labeled with a detection antibody that is capable of being combined with the target material on surfaces thereof, and which catalyze an oxidation reaction of the chromogenic substrate.

In order to accomplish the above objects, the present invention also provides a method of detecting a target material. The method includes detecting the target material using a lateral-flow immunochromatographic chip, which includes a conjugation unit including enzyme-mimic inorganic nanoparticles labeled with a detection antibody, and amplifying a detection signal using a chromogenic substrate and the enzyme-mimic inorganic nanoparticles.

The enzyme-mimic inorganic nanoparticles are combined with the target material, which is included in a liquid sample including the detection antibody for labeling, in the conjugation unit, and move together with the liquid sample to a reaction unit to combine the detection material, which is fixed to a predetermined portion of the reaction unit, and the target material to thus fix the materials to the predetermined portion of the reaction unit, thereby exhibiting the detection signal. The enzyme-mimic inorganic nanoparticles, which are fixed to the reaction unit, are used to oxidize the chromogenic substrate, thereby amplifying the detection signal. However, the enzyme-mimic inorganic nanoparticles are not theoretically limited thereto. All surfaces of the enzyme-mimic inorganic nanoparticles are exposed to the reaction, unlike the protein enzyme, which has sites that are capable of reacting with the substrate and are mostly covered, in the enzyme-substrate reaction for use in a signal amplification process in a conventional immunoassay. Accordingly, the reaction efficiency of the enzyme-mimic inorganic nanoparticles is very high, thus significantly improving a chromogenic characteristic. Further, the protein enzyme is significantly influenced by the surrounding environment (temperature, pH, and the like) due to the characteristic of proteins. However, the activity of the inorganic nanoparticles may be much more stable, and may be maintained over a long period of time.

In the present invention, the lateral-flow immunochromatographic chip means a kit that exhibits a chromogenic signal so as to enable identification with the naked eye during detection, by moving the liquid sample including the target material through a porous medium to react the target material with the fixed detection material.

In an embodiment of the present invention, the lateral-flow immunochromatographic chip may include a sample unit, to which the sample including the target material is fed, the conjugation unit (conjugation pad), which includes the monodispersed enzyme-mimic inorganic nanoparticles labeled with the detection antibody, a measurement unit, to which the detection material capable of being combined with the target material combined with the inorganic nanoparticles is fixed, a control unit for checking for errors, and an absorption unit absorbing the liquid sample using a capillary phenomenon.

The sample unit, the reaction unit, the measurement unit, the control unit, and the absorption unit of the assay kit of the lateral-flow immunochromatographic chip may be connected to each other via fine tubes or membranes. The membrane may include a porous natural or synthetic material, and may be nitrocellulose, without being limited thereto.

The enzyme-mimic inorganic nanoparticles mean a material that catalyzes the chemical reaction of various substrates, like the protein enzyme.

The term “chromogenic substrate” means a material that undergoes a chromogenic change before and after the reaction caused by the catalytic action of the enzyme-mimic nanoparticles.

It is understood that the terms “chromogenic change” and “amplification” mean one or more of the expression of colors, a change in chromogenic wavelength, and a change in chromogenic strength.

The term “target material” may be any one selected from the group consisting of antigen proteins, ligands, DNA, environmental hormones, environmental contaminants, and viruses, without limitation thereto, as long as the target material is a material capable of being combined with the detection antibody.

The term “detection antibody” means an antibody capable of being combined with the target material, Fab, which is a fragment of the antibody, or an antibody recombinant material. Such combination may be either chemical combination or physical combination.

The term “detection material” means a material that is capable of being combined with the target material or with the reaction material, which is combined with the target material. Such combination may be either chemical combination or physical combination, but physical combination, which is capable of being performed without a separate chemical reaction, is preferable. Examples of the reaction material may include an antibody, Fab, which is a fragment of an antibody, or recombinant scFv. The reaction material may be a receptor or a fragment of a receptor.

It is understood that the term “enzyme-substrate reaction” includes the reaction of the substrate, which is catalyzed by the enzyme, and the reaction of the chromogenic substrate, which is catalyzed by the enzyme-mimic nanoparticles.

In the present invention, the nanoparticles mean predetermined nano-sized particles having a diameter of less than 1000 nm. In some embodiments, the nanoparticles are less than 300 nm in diameter, based on the definition from the National Science Foundation. In some exemplary embodiments, the nanoparticles are less than 100 nm in diameter based on the definition from the National Institutes of Health. In preferable embodiments of the present invention, the nanoparticles are 10 to 90 nm in diameter.

In the present invention, the inorganic nanoparticles mean nanoparticles that include inorganic components. The inorganic materials may be inorganics, oxides of inorganic materials, or inorganic complexes, for example, complex materials which include metals, as well as nonmetals, ceramics, plastics, polymers, biological materials, semiconductors, or quantum dots. The complex materials may be, for example, particles which include nonmetallic nucleus material, such as ceramics or polymers, and which are coated with inorganic materials, and reactive functional groups or molecular sieves may be present on the surfaces of the inorganic particles.

In the present invention, the enzyme-mimic inorganic nanoparticles are inorganic nanoparticles, which are used to oxidize the chromogenic substrate, and are preferably particles including iron oxide (Fe₃O₄), platinum (Pt), graphene oxide, V₂O₃, and mixtures or alloys thereof.

In the present invention, the enzyme-mimic inorganic nanoparticles act as an oxidation catalyst to oxidize the chromogenic substrate and then precipitate and insolubilize the chromogenic substrate at a position at which the inorganic nanoparticles are present to thus exhibit a predetermined color, thereby amplifying the detection signal so that the detection signal is recognized by the naked eye.

In the preferable embodiment of the present invention, when the inorganic nanoparticles are peroxidase-mimetic inorganic nanoparticles, the chromogenic substrate may be 3-amino-9-ethylcarbazole (AEC).

In the present invention, functional groups for fixing the detection antibody may be present on the surfaces of the inorganic nanoparticles. The presence of functional groups on the surfaces of the inorganic nanoparticles is a means for fixing the detection antibody, and is preferably a carboxylic acid group, exerting a covalent bond or ionic electric attraction force. For this, an amine group (—NH₂), which is abundant in an IgG antibody, may be used.

The inorganic nanoparticles according to the present invention are preferably monodispersed so as to be included in a liquid to thus move to the conjugation unit of the lateral-flow immunochromatographic chip through a capillary tube having a diameter of ones of micrometers.

Monodispersity is an index indicating the degree of uniformity of the sizes and structures of the nanoparticles, and indicates the degree of substantial uniformity.

Monodispersed inorganic nanoparticles may be manufactured based on the document [(J. Am. Chem. Soc., Shouheng Sun et al., Monodispersed MFe₂O₄ (M=Fe, Co, Mn) nanoparticles: 2004, Vol. 126, pp. 273-279], hereby introduced as a reference in the present invention.

In the embodiment of the present invention, the monodispersed inorganic nanoparticles may be manufactured using a synthesis method for improving monodispersity using a surfactant in an organic solvent.

The detection antibody is fixed to the surfaces of the inorganic nanoparticles and is then dispersed in a normal saline solution, including sucrose and bovine serum albumin, to thus be uniformly absorbed in the conjugation unit including glass fibers, followed by drying. Since the glass fibers have low affinity to proteins, when the dried antibody-inorganic nanoparticles meet an analyte solution between the glass fibers, the nanoparticles may be hydrated and then easily move to a test pad.

In order to detect the analyte, the body fluid including the analyte may be allowed to flow into the lateral-flow immunochromatographic chip and cause an enzyme-substrate reaction using a precipitable and insoluble substrate, thereby amplifying a chromogenic signal to thus successfully improve detection sensitivity.

### Advantageous Effects

The high-sensitivity lateral-flow immunochromatographic chip according to the present invention adopts a method of amplifying a signal using an enzyme-substrate reaction. The amplified signal may be simply observed with the naked eye in the field using a chromogenic substrate oxidized to form a precipitate.

Further, since enzyme-mimic inorganic nanoparticles are used, a detection system may be more stably maintained than that of a conventional enzyme including proteins. Accordingly, a small amount of an analyte may be detected with high sensitivity from the body fluid to thus enable the early diagnosis and initial countermeasure of acute diseases or cancers. The detection system may be stably maintained, thus being applicable to a diagnostic tool to be provided as relief goods in civil war or disaster areas.

### DESCRIPTION OF DRAWINGS

FIG. 1 shows the manufacture of a lateral-flow immunochromatographic chip, which includes inorganic nanoparticles, and the attachment of an antibody to the inorganic nanoparticles, which are synthesized using an analyte detection method based on an enzyme-substrate reaction;

FIG. 2 shows the manufacture of the lateral-flow immunochromatographic chip, which includes the inorganic nanoparticles, and the detection of the analyte using the lateral-flow immunochromatographic chip and the amplification of a chromogenic signal using the enzyme-substrate reaction according to the analyte detection method based on the enzyme-substrate reaction;

FIG. 3 is a mimetic view showing a lateral-flow immunochromatographic assay using nanoparticles, to which a pure mouse IgG antibody having no detection function is fixed, when the analyte detection function of the lateral-flow immunochromatographic chip, which includes the inorganic nanoparticles, is verified;

FIG. 4 shows the result of a reaction suitability test of two types of AEC and DAB substrates with the inorganic nanoparticles when the analyte detection function of the lateral-flow immunochromatographic chip, which includes the inorganic nanoparticles, is verified;

FIG. 5 shows the images of the chromogenic signal, which is amplified over a time due to the enzyme-substrate reaction, during a detection sensitivity test using hCG;

FIG. 6 shows a standard curve of the detection sensitivity test using hCG, depending on a concentration of hCG; and

FIG. 7 shows a standard curve of the detection sensitivity test using hCG, depending on an intensity ratio value amplified over an enzyme-substrate reaction time.

### MODE FOR INVENTION

A better understanding of the present invention may be obtained through the following Examples which are set forth to illustrate, but are not to be construed to limit the present invention.

### EXAMPLES

### Example 1

### Synthesis of Inorganic Nanoparticles and Surface Fixing of Antibody

### 1-1 Synthesis of Inorganic Nanoparticles

Iron oxide nanoparticles were synthesized using a coprecipitation process. For synthesis thereof, 0.4 g of ferrous chloride and 1.1 g of ferric chloride were added to 20 mL of distilled water, and the temperature was increased to 80° C. under the flow of argon gas. 5 mL of a 28% ammonium hydroxide solution was added at 80° C. to induce a reaction for one hr, and the temperature was then reduced to room temperature. Subsequently, washing with ethanol and distilled water was repeated five times using a magnet.

Platinum nanoparticles were synthesized using a modified synthesis method including conventional seed growing. The procedure therefore will be broadly described below in brief. 18 mL of 0.2% chloroplatinic acid hexahydrate was added to 232 mL of boiling water and, after 1 min, 1% sodium citrate and 0.05% citric acid were each added in an amount of 5.5 mL. A solution including fresh 0.08% sodium borohydrate, 1% sodium citrate, and 0.05% citric acid was added in an amount of 2.75 mL after 30 sec. Platinum nanoparticle seeds having a diameter of 5 nm, obtained after the reaction was performed for 10 min, were used to synthesize platinum nanoparticles. After 10 mL of the seed solution and 90 mL of distilled water were mixed, 0.05 mL of 0.4M chloroplatinic acid hexahydrate (particles having a diameter of 30 nm were added in an amount of 0.5 mL), 1% sodium citrate, and 0.5 mL of 1.25% L-ascorbic acid were added, heated at a rate of 10° C./min to a boiling point, maintained for 30 min, and cooled. The particles were precipitated using a centrifuge for 20 min (8000 rpm), and washing was then repeated with ethanol and distilled water three times.

### 1-2 Labeling of Inorganic Nanoparticles with Detection Antibody

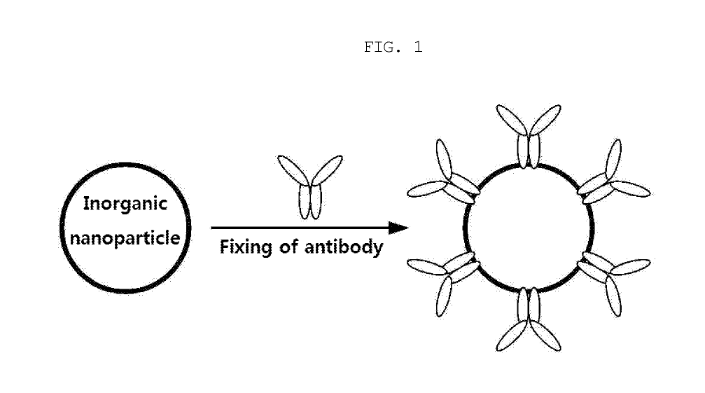

In order to fix the detection antibody to the monodispersed inorganic nanoparticles transported to an aqueous system, two materials were mixed in water at a ratio of 1:1 (a final concentration of 0.5 mg/mL). The two materials were sufficiently dispersed in water and then subjected to a fixing reaction at 4° C. for 24 hrs (FIG. 1). After the reaction, the antibodies, which were not attached to the surfaces of the inorganic nanoparticles, were removed using a centrifuge (14,000 rpm, 15 min, 4° C.), and then the inorganic nanoparticles labeled with the detection antibody were stored in a normal saline solution.

### Example 2

### Assembly of Lateral-Flow Immunochromatographic Chip

### 2-1 Storage of Inorganic Nanoparticles Labeled with Detection Antibody and Assembly

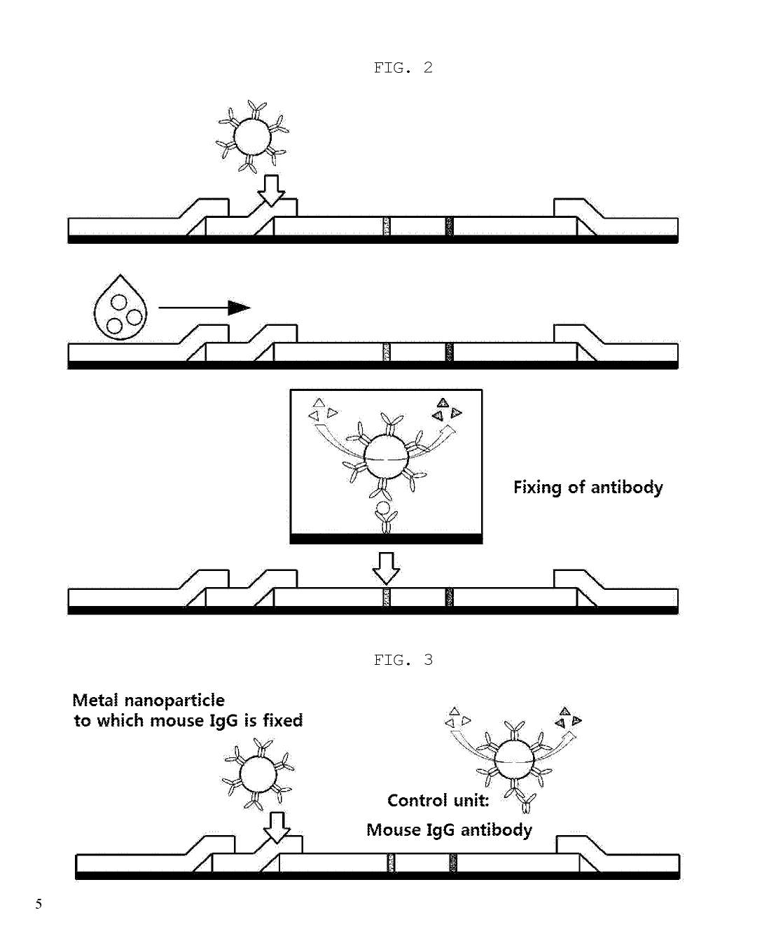

The inorganic nanoparticles labeled with the detection antibody in Example 1-2 were dried and stored in a conjugation unit, which was positioned beside a sample pad in the lateral-flow immunochromatographic chip (step 1 of FIG. 2). The conjugation unit includes glass fibers, and glass fibers have poor compatibility with proteins. Accordingly, when a solution flows out from a sample pad, a dried detection reagent, which is present in the conjugation unit, may be hydrated and easily move to a test pad. In order to improve hydration ability, sucrose, which was a saccharide component, was added in an amount of 10 wt %, and the inorganic nanoparticles labeled with the detection antibody were uniformly dispersed in a normal saline solution, including 3 wt % of bovine serum albumin for reducing a non-specific reaction, at a concentration of 1 mg/mL, and then moved to a glass pad. A total of 8 μg of the nanoparticles were stored in a single chip, and the sample was reacted with all of the nanoparticles to be detected. The glass pad storing the inorganic nanoparticles in the solution was sufficiently dried in a vacuum environment, and was then positioned between the sample pad and the test pad, thereby completing the chip.

### Example 3

### Verification of Analyte Detection Function of Lateral-Flow Immunochromatographic Chip Including Inorganic Nanoparticles

### 3-1 Confirmation of Fixing State of Detection Antibody Using Anti-Mouse Antibody Section

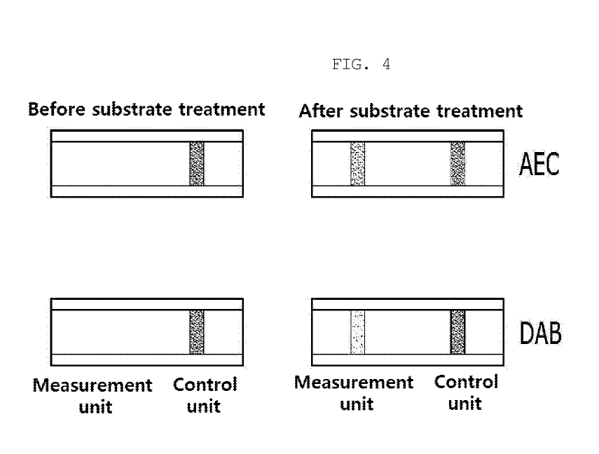

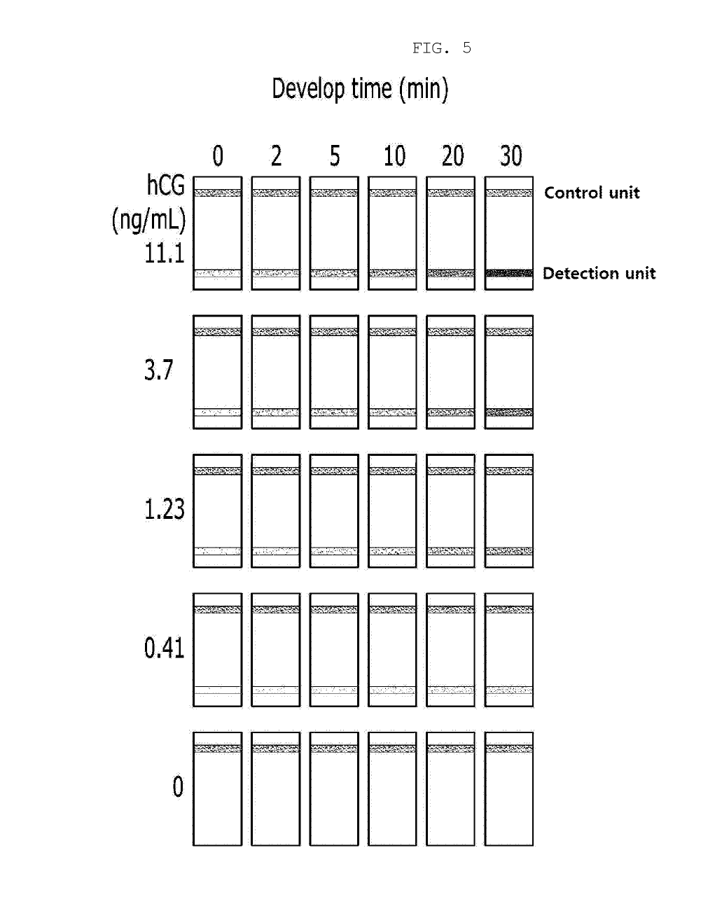

In order to confirm the state of the detection antibody, which was reacted with the carboxyl group on the surfaces of the inorganic nanoparticles to be fixed, a test was performed using the anti-mouse antibody section positioned in the control unit of a lateral-flow immunochromatographic chip (FIG. 3). Pure mouse IgG proteins having no detection function were fixed to the surface of the inorganic nanoparticles as in Example 1-2, the resulting inorganic nanoparticles were put into the chip as in Example 2-1, and 100 μl of a normal saline solution was made to flow through a sample pad as in step 2 of FIG. 2. Accordingly, from the image of FIG. 4 before substrate treatment, it could be confirmed that mouse IgG was caught by the anti-mouse antibody in the control unit to produce a chromogenic signal, and the chromogenic signal was visible to the naked eye. Since the inorganic nanoparticles labeled with the antibody were also caught, the chromogenic signal exhibited the intrinsic color of the inorganic nanoparticles.

### 3-2 Selection of Substrate Capable of Reacting with Inorganic Nanoparticles

The substrate, which is to be applied to the lateral-flow immunochromatographic chip of the present inventor, must react with an enzyme to form a precipitate to thus exhibit a color. Representative examples thereof include a substrate, which is reacted with a peroxidase to form a precipitate to thus exhibit a color such as 3-amino-9-ethylcarbazole (AEC) and 3,3′-diaminobenzidine (DAB). AEC is a substrate which exhibits a yellow color before oxidation but is changed to thus exhibit a red color after the reaction. DAB is a substrate which exhibits an orange color before oxidation but is changed to thus exhibit a brown color after the reaction. First, the inorganic nanoparticles were positioned in the control unit on the chip using mouse IgG as in Example 3-1, and treatment was performed sequentially using 100 μl of each of two types of substrate. As a result, the substrate that reacted with the inorganic nanoparticles developed by the present inventor was AEC, and from FIG. 4, it could be confirmed that the control unit exhibited a red color after substrate treatment.

### Example 4

### Measurement of Sensitivity of Lateral-Flow Immunochromatographic Chip Using Human Chorionic Gonadotropin (hCG) Analyte

### 4-1 Fixing of Anti-hCG Antibody

In order to fix a detection antibody for detecting hCG to the surfaces of waterborne platinum nanoparticles (30 nm) after synthesis, a fixing process using secondary combination between two materials was selected and applied. After final synthesis, a β-hCG monoclonal antibody, which was the detection antibody, was added at a concentration of 0.1 mg/mL to the platinum nanoparticles (0.5 mg/mL) dispersed in water (deionized water), sufficiently mixed, and reacted in a rotator at 4° C. for 24 hrs. After the reaction, washing was repeated twice using a normal saline solution (14,000 rpm, 15 min, 4° C.), and the platinum nanoparticles, to which the antibody was fixed, were finally stored in a conjugation unit storage solution (10 wt % sucrose, 0.1 wt % Tween 20, 3 wt % BSA, PBS) or a preservation solution (1% BSA, PBS) at a concentration of 1 mg/mL.

### 4-2 Detection of hCG Using Enzyme-Substrate Reaction

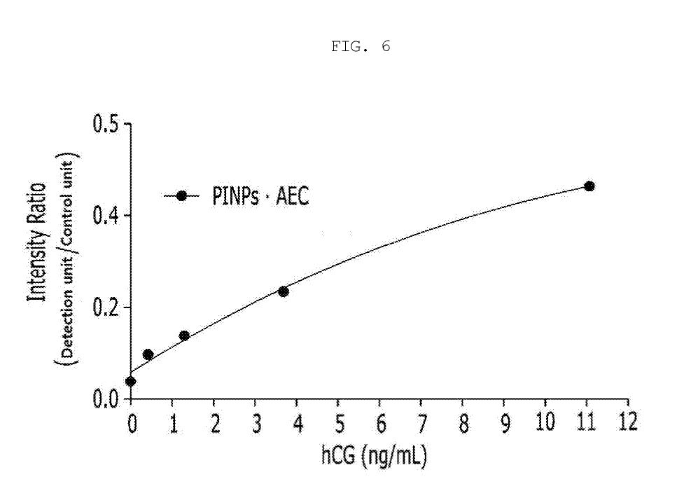

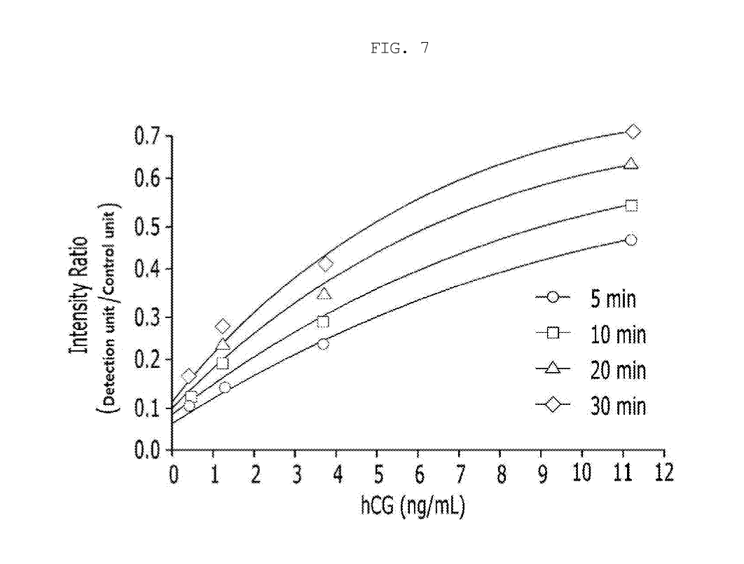

The detection sensitivity, which was amplified due to the enzyme-substrate reaction of inorganic nanoparticles and AEC, was measured using the lateral-flow immunochromatographic chip, manufactured in Example 4-1, and was used to detect hCG. A commercial dipstick for use in detection of hCG has a sensitivity of 25 mIU/mL (3.7 ng/mL). The concentration of hCG was set to the range of 11.1 to 0.41 ng/mL using the dipstick, and a standard detection curve was drawn within the aforementioned range. A predetermined concentration of hCG was mixed with a normal saline solution, and the mixture was allowed to flow to a sample pad. The mixture was allowed to flow through a test pad for about 10 min, thus being captured by the capture antibody of a detection unit. In order to create an environment in which a reaction with a substrate occurs, the test pad was sufficiently washed with a sodium acetate solution having a pH of 4.5, and 100 μl of an AEC substrate solution was allowed to drip on the detection unit and a control unit so as to prevent the pad from drying, thereby causing an enzyme-substrate reaction. As the result of the enzyme-substrate reaction, red signals were observed at a control line and a test line with the passage of time. Only the color of the inorganic nanoparticles was not observed before the reaction with the substrate at a concentration of 1.23 ng/mL or less. After substrate treatment, the chromogenic signal was amplified over time to gradually form a red band. When only a normal saline solution (0 ng/mL) including no hCG was applied, the red color was not observed at the test line, even after the passage of time. Accordingly, it could be confirmed that all chromogenic signals shown in FIG. 5 were apparently values of detected hCG. FIG. 6 shows a graph obtained by measuring chromogenic values of a detection unit using an image assay. The standard curve value, depending on the hCG concentration, was defined as the ratio obtained by dividing the signal value of the detection unit by the signal value of the control unit in each chip. FIG. 7 shows that the intensity of the detection signal is increased as the time of the reaction with the substrate is increased.

### INDUSTRIAL APPLICABILITY

The high-sensitivity lateral-flow immunochromatographic chip according to the present invention adopts a method of amplifying a signal using an enzyme-substrate reaction. The amplified signal may be simply observed with the naked eye in the field using a chromogenic substrate oxidized to form a precipitate.

Further, since enzyme-mimic inorganic nanoparticles are used, a detection system may be stably maintained, compared to a conventional enzyme including proteins. Accordingly, a small amount of an analyte from body fluid may be detected with high sensitivity to thus enable the early diagnosis of acute diseases or cancers and initial countermeasures therefor. The detection system may be stably maintained, thus being applicable to a diagnostic tool to be provided as relief goods in civil war or disaster areas.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A lateral-flow immunochromatographic chip comprising:
a conjugation unit that includes enzyme-mimic inorganic nanoparticles including a detection antibody.

2. The lateral-flow immunochromatographic chip of claim 1, wherein the inorganic nanoparticles are combined with a target material to be fixed to a reaction unit, and are reacted with a chromogenic substrate to amplify a detection signal.

3. The lateral-flow immunochromatographic chip of claim 1, wherein the inorganic nanoparticles are monodispersed inorganic nanoparticles.

4. The lateral-flow immunochromatographic chip of claim 1, wherein the inorganic nanoparticles include iron oxide and platinum.

5. The lateral-flow immunochromatographic chip of claim 1, wherein the detection antibody is reacted with a carboxyl group, which is present on surfaces of the inorganic nanoparticles, to be combined with the inorganic nanoparticles.

6. The lateral-flow immunochromatographic chip of claim 1, wherein the inorganic nanoparticles include the detection antibody, which is combined with a detection material fixed to a support of the reaction unit.

7. The lateral-flow immunochromatographic chip of claim 1, wherein the enzyme-mimic inorganic nanoparticles are an oxidation catalyst of the chromogenic substrate.

8. The lateral-flow immunochromatographic chip of claim 7, wherein the chromogenic substrate is precipitated and insolubilized using the enzyme-mimic inorganic nanoparticles.

9. The lateral-flow immunochromatographic chip of claim 8, wherein the chromogenic substrate is 3-amino-9-ethylcarbazole.

10. The lateral-flow immunochromatographic chip of claim 1, wherein the lateral-flow immunochromatographic chip includes a sample unit, to which a sample including the target material is fed, the conjugation unit (conjugation pad), which includes the monodispersed enzyme-mimic inorganic nanoparticles labeled with the detection antibody, a measurement unit, to which a detection material capable of being combined with the target material combined with the inorganic nanoparticles is fixed, a control unit for checking for errors, and an absorption unit absorbing a liquid sample using a capillary phenomenon.

11. A method of detecting a target material, the method comprising:
detecting the target material using the lateral-flow immunochromatographic chip according to claim 1; and
reacting a chromogenic substrate with enzyme-mimic inorganic nanoparticles to amplify a detection signal.

12. Enzyme-mimic inorganic nanoparticles which are labeled with a detection antibody capable of being combined with a target material on surfaces thereof and which are used to oxidize a chromogenic substrate.

13. The enzyme-mimic inorganic nanoparticles of claim 12, wherein the inorganic nanoparticles include iron oxide, platinum, or a mixture thereof.

14. The enzyme-mimic inorganic nanoparticles of claim 12, wherein the inorganic nanoparticles are physically combined with the detection antibody to be labeled with the detection antibody.

15. The enzyme-mimic inorganic nanoparticles of claim 12, wherein the inorganic nanoparticles are monodispersed inorganic nanoparticles.

16. The enzyme-mimic inorganic nanoparticles of claim 12, wherein the chromogenic substrate is precipitated and insolubilized using the enzyme-mimic inorganic nanoparticles.

17. A method of manufacturing a lateral-flow immunochromatographic chip, the method comprising:
synthesizing monodispersed enzyme-mimic inorganic nanoparticles;
labeling the inorganic nanoparticles with a detection antibody; and
positioning the inorganic nanoparticles, which are labeled with the detection antibody, in a conjugation unit of the lateral-flow immunochromatographic chip.

18. The method of claim 17, wherein the inorganic nanoparticles are synthesized using iron oxide and platinum.

19. The method of claim 17, wherein sizes of the inorganic nanoparticles are uniformly controlled using a surfactant in an organic solvent environment to thus monodisperse the synthesized inorganic nanoparticles.

20. The method of claim 17, wherein the inorganic nanoparticles are substituted with a carboxylic acid group on surfaces thereof, and the substituted inorganic nanoparticles and the detection antibody are uniformly dispersed in a normal saline solution to be fixed.
